(19) United States
(12) Patent Application Publication
(10) Pub. No.: US 2016/0014987 A1
(43) Pub. Date: Jan. 21, 2016

(54) Breeding Method of Plant Induced Mutation of Breeding Target Plant

(71) Applicant: Changyi Cao, (US)

(72) Inventor: Changyi Cao, Zhengzhou (CN)

(21) Appl. No.: 14/867,012

(22) Filed: Sep. 28, 2015

Related U.S. Application Data
(63) Continuation-in-part of application No. PCT/CN2014/082344, filed on Jul. 16, 2014.

(30) Foreign Application Priority Data
Jul. 25, 2013 (CN) 201310317334.7

(51) Int. Cl. A01H 1/02

(52) U.S. Cl. CPC A01H 1/02

(57) ABSTRACT

A breeding method for variation of target plant for plant induced breeding comprises the following steps: 1) breeding an induced plant seedling, and using a bud point or a node on the induced plant seedling as an implanting notch point; 2) selecting a breeding target plant seedling for standby; 3) notching on the implanting notch point; 4) implanting the breeding target plant seedling into a notch; 5) fixing the breeding target plant and an induced plant, covering the same with wet soil, and compacting; 6) shading and curing, and sprinkling water per day; and 7) after 45 to 60 days, cutting off both sides of the induced plant notch by 5 to 10 cm; and transplanting a section of cut induced plant stem together with soil around the breeding target plant and the notch to obtain. The above-mentioned method can realize mutation breeding including among plants on same edge, near edge or far edge, is suitable for most of plants and has the advantages of high success rate of breeding, high mutation probability, favorable comprehensive mutation performance, diversified mutation performance, quick stability and the like.

### CROSS-REFERENCE TO RELATED APPLICATIONS

This application is a continuation-in-part of International Patent Application No. PCT/CN2014/082344 with an international filing date of Jul. 16, 2014, designating the United States, now pending, and further claims priority benefits to Chinese Patent Application No. 201310317334.7 filed Jul. 25, 2013. The contents of all of the aforementioned applications, including any intervening amendments thereto, are incorporated herein by reference.

### TECHNICAL FIELD

The present invention relates to a breeding method for variation of target plant for plant induced breeding, and belongs to the field of plant breeding.

### BACKGROUND OF THE PRESENT INVENTION

With the continuous increase of the world's population, the need for grain is also continuously increased. Therefore, there is an urgent need for breeding a new variety of crops to enhance the quality and the yield level of traditional crops. Meanwhile, with the increase of living standard, people have higher and higher requirement for living quality and are dissatisfied with the existing plant variety. Therefore, plant breeding has become a hot subject researched by international scholars.

Currently common breeding methods include selective breeding, hybridization breeding, mutation breeding (mutation breeding is also called physical mutation breeding and chemical mutation breeding), biotechnology breeding, etc. But these breeding means have the following defects to different extents: 1. long breeding period: it generally takes several years or even several decades to cultivate hybrid rice; for example, Xu Zhaoxi takes nearly 20 years from selecting, purifying, discarding, comparing varieties, etc. to cultivate panax ginseng new variety “Panax No. 1”; 2. low success rate of breeding; and 3. low breeding predictability; a breeder often breeds tens of or even hundreds of hybridized combinations, but the comprehensive performance of new hybridized varieties is poor. Therefore, there is an urgent need for seeking a new plant breeding method.

### SUMMARY OF THE PRESENT INVENTION

The purpose of the present invention is to provide a breeding method for variation of target plant for plant induced breeding, capable of realizing mutation breeding including among plants on same edge, near edge or far edge.

To realize the above purpose, the technical solution adopted in the present invention is to provide a breeding method for variation of target plant for plant induced breeding, comprising the following steps:

1) breeding an induced plant seedling, and using a bud point or a node on the induced plant seedling as an implanting notch point;

2) selecting a breeding target plant seedling for standby;

3) notching on the implanting notch point;

4) cutting the lower part of the breeding target plant seedling into a shape consistent with a notch and then, implanting the breeding target plant seedling into the notch;

5) fixing the breeding target plant and an induced plant, covering the same with wet soil, and compacting;

6) shading and curing, and sprinkling water per day; and

7) after 45 to 60 days, cutting off both sides of the induced plant notch by 5 to 10 cm; and digging out and transplanting a section of cut induced plant stem together with soil around the breeding target plant and the notch to obtain.

The implanting notch point in step 1) is a bud point or a node in a position 3 to 10 cm away from the root of the induced plant seedling.

The breeding target plant seedling in step 2) has a height of 3 to 10 cm.

In step 3), on the implanting notch point, notching is performed along the growth direction of the induced plant stem.

An implanting environment in step 4) has a temperature of 25 to 30° C. and a relative humidity of 75% to 85%.

The implanting time in step 4) is at 1 to 3 a.m., 8 to 11 a.m. or 3 to 6 p.m.

The water content of the wet soil in step 5) is 75% to 85%.

The sprinkling of water in step 6) is 8 to 15 times per day.

The present invention uses a plant to be induced as a breeding target plant, and selects a plant with obvious character features, obvious mutation effect and easy operation of plant mutation as a mutation plant, and performs mutation breeding on the breeding target plant.

Currently realized breeding target plant comprises: wheat, barley, soybean, corn, rice, millet, sorghum, oat, wild oat, mung bean, sesame, rapeseed, peanut, cotton, sweet potato, potato, Chinese yam, pumpkin, chili, melon, watermelon, Chinese rose, platycodon root, etc.

It has been proved that the induced plant with good induction effect comprises: potato, sweet potato, taro, snow lotus, peanut, corn, sorghum, soybean, barley, sesame, rapeseed, Chinese yam, ginseng, fleeceflower root, radix rehmanniae, honeysuckle, radix isatidis, sunflower, ricinus communis, dahlia, peony, paeonia lactiflora pall, Chinese rose, impatiens balsamina, walnut, moringa tree, pomegranate, fig, loquat, wild jujube tree, Chinese toon tree, bamboo, sugarcane, portulaca oleracea L, cactus, aloe, wormwood, pumpkin, calabash, dishcloth gourd, bitter gourd, kidney bean, melon, watermelon, zinger officinale roscoe, tomato, eggplant, three-coloured amaranth, lettuce, purple perilla, chili, strawberry, alfalfa, conyza bonariensis, lactuca tatarica, blackberrylily rhizome grass, etc.

The present invention has the following advantages:

1) The mutation breeding among plants on same edge, near edge or far edge can be realized; the breeding target plant and the induced plant have wide selection range; the present invention is applicable to most of plants; and the mutation breeding of any plant can be realized to a certain extent.

2) The implanting survival rate is above 90%, the mutation rate is above 30%, the ideal variation rate is above 10%, the mutation strength is high, the diversity is good, the stability is fast, and the comprehensive mutation performance is good.

3) The success probability of breeding is high and can reach more than 90%, and the disadvantage of low success probability in a conventional breeding method is overcome.

4) The breeding period is shortened by ⅓ to ½ (taking wheat as an example, the conventional breeding generally needs about 10 years, and only 3 to 5 year by adopting the method of the present invention), the breeding efficiency is high, and multi-variety, factory and batch mutation breeding can be realized.

5) The breeding cost is low and the comprehensive cost is reduced by times.

6) The technology is simple, the operation performance is strong and the popularization is convenient.

### DETAILED DESCRIPTION OF THE PRESENT INVENTION

### Embodiment 1

In the embodiment, the sweet potato is used as a breeding target plant and the fleece flower root is used as an induced plant for implementing the implanting of a fission for performing the mutation breeding of the sweet potato, comprising the following steps:

1) planting a fleece flower root seed until a seedling thereof grows to 30 to 50 cm, and using a bud point of a fleece flower root stem in a position 3 to 5 cm away from the upper part of the root as an implanting notch point;

2) selecting a sweet potato seedling having a seedling height of 3 to 5 cm for standby;

3) notching on the implanting notch point along the growth direction of the fleeceflower root stem, wherein the size and the depth of the notch are determined according to the thickness of the sweet potato seedling;

4) cutting the lower part of the sweet potato seedling into a shape consistent with a notch and then, implanting the sweet potato seedling into the notch under the conditions of 2 a.m., a temperature of 25° C. and a relative humidity of 85%;

5) fixing the sweet potato seedling and the fleeceflower root seedling with a film, covering the same with wet soil having the water content of 85%, and compacting;

6) shading and curing, and sprinkling water in a mist shape with an electric atomizer for 8 to 10 times per day;

7) after 10 to 15 days, growing 1 to 2 autogenetic roots by the sweet potato seedling from the upper part of the notch; up to 45 days, growing 3 to 5 autogenetic roots by the sweet potato seedling; and independently growing; and

8) cutting off both sides of the notch of the fleeceflower root seedling by 10 cm, digging out a section of cut fleeceflower root stem together with soil around the sweet potato seedling and the notch and transplanting to a prepared breeding field; and proceeding with curing growth.

A small sweet potato born by the sweet potato of the same year is used as a seed. In the second year, breeding and planting are performed; after the sweet potato is harvested, it is found from comparison with a non-mutated sweet potato that a block of the sweet potato through the adoption of the mutation breeding of the present invention generates obvious apparent variation; the shape is like the shape of the fleeceflower root; three, four and five sweet potatoes as a continuum form different strings; and the taste is also different from the non-mutated sweet potato. It is proved from an experiment that the implanting survival rate is above 90%, the mutation rate is above 30%, the ideal variation rate is above 10% and the success rate of breeding is above 90%.

### Embodiment 2

In the embodiment, the soybean is used as a breeding target plant and the potato is used as an induced plant for implementing the implanting of a fission for performing the mutation breeding of the soybean, comprising the following steps:

1) planting a potato seed until a seedling thereof grows to 30 to 50 cm, and using a bud point of a potato stem in a position 3 to 5 cm away from the upper part of the root as an implanting notch point;

2) selecting a soybean seedling having a seedling height of 3 to 5 cm for standby;

3) notching on the implanting notch point along the growth direction of the potato stem, wherein the size and the depth of the notch are determined according to the thickness of the soybean seedling;

4) cutting the lower part of the soybean seedling into a shape consistent with a notch and then, implanting the soybean seedling into the notch under the conditions of 1 a.m., a temperature of 25° C. and a relative humidity of 75%;

5) fixing the soybean seedling and the potato seedling with a film, covering the same with wet soil having the water content of 85%, and compacting;

6) shading and curing, and sprinkling water in a mist shape with an electric atomizer for 10 to 15 times per day;

7) after about 15 days, growing autogenetic roots by the soybean seedling from the upper part of the notch, up to about 45 days, growing 5 to 7 autogenetic roots by the soybean seedling; and independently growing; and

8) cutting off both sides of the notch of the potato seedling by 10 cm, digging out a section of cut potato stem together with soil around the soybean seedling and the notch and transplanting to a prepared breeding field; and proceeding with curing growth.

Ten seeds are born by the soybean in the same year. In the second year, 6 seeds are sown and survived; F1 generation is harvest; and an experimental field is sown. Compared with the non-mutated soybean seedling, the soybean seedling is obviously varied; the mutated soybean has a plant height of 80 to 90 cm, and the non-mutated soybean has a plant height of 70 to 75 cm; after the soybean is harvested, obvious fertile features are exhibited: compact plant type and more pods wherein an average of 180 grains can be reached per plant. After planting in a peasant field, the yield per mu achieves more than 290 kilograms, while the yield per mu of the non-mutated soybean is 160 to 200 kilograms. Detected by Supervision and Inspection Center of Agricultural Products of the Ministry of Agriculture, the protein content achieves more than 44% higher than that of the non-mutated soybean by more than 3 percentage points and higher than American transgenic soybean by 9 percentage points. It is proved from an experiment that the implanting survival rate is above 90%, the mutation rate is above 30%, the ideal variation rate is above 10% and the success rate of breeding is above 90%.

### Embodiment 3

In the embodiment, the soybean is used as a breeding target plant and the peanut is used as an induced plant for implementing the implanting of a fission for performing the mutation breeding of the soybean, comprising the following steps:

1) planting a peanut seed until a seedling thereof grows to 30 to 50 cm, and using a bud point of a peanut stem in a position 3 to 5 cm away from the upper part of the root as an implanting notch point;

2) selecting a soybean seedling having a seedling height of 3 to 5 cm for standby;

3) notching on the implanting notch point along the growth direction of the peanut stem, wherein the size and the depth of the notch are determined according to the thickness of the soybean seedling;

4) cutting the lower part of the soybean seedling into a shape consistent with a notch and then, implanting the soybean seedling into the notch under the conditions of 1 a.m., a temperature of 25° C. and a relative humidity of 75%;

5) fixing the soybean seedling and the peanut seedling with a film, covering the same with wet soil having the water content of 80%, and compacting;

6) shading and curing, and sprinkling water in a mist shape with an electric atomizer for 10 to 15 times per day;

7) after about 15 days, growing autogenetic roots by the soybean seedling from the upper part of the notch; up to about 45 days, growing 5 to 7 autogenetic roots by the soybean seedling; and independently growing; and

8) cutting off both sides of the peanut seedling notch by 10 cm; digging out a section of cut peanut stem together with soil around the soybean seedling and the notch and transplanting to a prepared breeding field; and proceeding with curing growth.

The soybean seed of the same year is sown in the second year, and cultivated high-oil soybean has an oil content of achieving 23% higher than the non-mutated soybean by 6 percentage points and higher than American transgenic soybean by 3 percentage points. It is proved from an experiment that the implanting survival rate is above 90%, the mutation rate is above 30%, the ideal variation rate is above 10% and the success rate of breeding is above 90%.

### Embodiment 4

In the embodiment, the cotton is used as a breeding target plant and the dahlia is used as an induced plant for implementing the implanting of a fission for performing the mutation breeding of the cotton, comprising the following steps:

1) planting a dahlia seed until a seedling thereof grows to 30 to 50 cm, and using a bud point of a dahlia stem in a position 6 to 10 cm away from the upper part of the root as an implanting notch point;

2) selecting a cotton seedling having a seedling height of 5 to 10 cm for standby;

3) notching on the implanting notch point along the growth direction of the dahlia stem, wherein the size and the depth of the notch are determined according to the thickness of the cotton seedling;

4) cutting the lower part of the cotton seedling into a shape consistent with a notch and then, implanting the cotton seedling into the notch under the conditions of 3 p.m., a temperature of 30° C. and a relative humidity of 80%;

5) fixing the cotton seedling and the dahlia seedling with a film, covering the same with wet soil having the water content of 75%, and compacting;

6) shading and curing, and sprinkling water in a mist shape with an electric atomizer for about 10 times per day;

7) after about 20 days, growing autogenetic roots by the cotton seedling from the upper part of the notch; up to 45-60 days, the cotton seedling being capable of independently growing; and

8) cutting off both sides of the dahlia seedling notch by 10 cm; digging out a section of cut dahlia stem together with soil around the cotton seedling and the notch and transplanting to a prepared breeding field; and proceeding with curing growth.

The cotton blossoms and seeds in the same year as a seed. In the second year, breeding and planting are performed; when the cotton blossoms and is picked, the cotton presents light red and is obviously different from white floss of the non-mutated cotton. It is proved from an experiment that the implanting survival rate is above 90%, the mutation rate is above 30%, the ideal variation rate is above 10% and the success rate of breeding is above 90%.

### Embodiment 5

In the embodiment, the cotton is used as a breeding target plant and the sunflower is used as an induced plant for implementing the implanting of a fission for performing the mutation breeding of the cotton, comprising the following steps:

1) planting a sunflower seed until a seedling thereof grows to 30 to 50 cm, and using a bud point of a sunflower stem in a position 6 to 10 cm away from the upper part of the root as an implanting notch point;

2) selecting a cotton seedling having a seedling height of 5 to 10 cm for standby;

3) notching on the implanting notch point along the growth direction of the sunflower stem, wherein the size and the depth of the notch are determined according to the thickness of the cotton seedling;

4) cutting the lower part of the cotton seedling into a shape consistent with a notch and then, implanting the cotton seedling into the notch under the conditions of 6 p.m., a temperature of 30° C. and a relative humidity of 80%;

5) fixing the cotton seedling and the sunflower seedling with a film, covering the same with wet soil having the water content of 80%, and compacting;

6) shading and curing, and sprinkling water in a mist shape with an electric atomizer for about 10 times per day;

7) after 20 days, growing autogenetic roots by the cotton seedling from the upper part of the notch; up to 45-60 days, the cotton seedling being capable of independently growing; and

8) cutting off both sides of the sunflower seedling notch by 10 cm; digging out a section of cut sunflower stem together with soil around the cotton seedling and the notch and transplanting to a prepared breeding field; and proceeding with curing growth.

The cotton blossoms and seeds in the same year as a seed. In the second year, breeding and planting are performed; when the cotton blossoms and is picked, the cotton presents light black and is obviously different from white floss of the non-mutated cotton. It is proved from an experiment that the implanting survival rate is above 90%, the mutation rate is above 30%, the ideal variation rate is above 10% and the success rate of breeding is above 90%.

### Embodiment 6

In the embodiment, the peanut is used as a breeding target plant and the juglans seedling is used as an induced plant for implementing the implanting of a fission for performing the mutation breeding of the peanut, comprising the following steps:

1) culturing a juglans seedling until the seedling grows to 5 to 10 cm, and using a bud point of a walnut seedling in a position 3 to 5 cm away from the upper part of the root as an implanting notch point;

2) selecting a peanut seedling having a seedling height of 3 to 5 cm for standby;

3) notching on the implanting notch point along the growth direction of the juglans stem, wherein the size and the depth of the notch are determined according to the thickness of the peanut seedling;

4) cutting the lower part of the peanut seedling into a shape consistent with a notch and then, implanting the peanut seedling into the notch under the conditions of 8 a.m., a temperature of 26° C. and a relative humidity of 83%;

5) fixing the peanut seedling and the juglans seedling with a film, covering the same with wet soil having the water content of 75%, and compacting;

6) shading and curing, and sprinkling water in a mist shape with an electric atomizer for 10 to 15 times per day;

7) after about 15 days, growing autogenetic roots by the peanut seedling from the upper part of the notch; up to about 50 days, growing 5 to 7 autogenetic roots by the peanut seedling; and independently growing; and

8) cutting off both sides of the walnut seedling notch by 5 cm; digging out a section of cut juglans stem together with soil around the peanut seedling and the notch and transplanting to a prepared breeding field; and proceeding with curing growth.

Eight seeds are born by the peanut in the same year. In the second year, 5 seeds are sown and survived; F1 generation is harvest; and an experimental field is sown. Compared with the non-mutated peanut seedling, the peanut seedling is obviously varied; the mutated peanut has a plant height of 40 to 50 cm, and the non-mutated peanut has a plant height of 30 to 35 cm; after the peanut is harvested, obvious fertile features are exhibited: more fruit and thin peel. After planting in a peasant field, the yield per mu of the peanut in spring sowing achieves more than 600 kilograms, while the yield per mu of the peanut in summer sowing is more than 450 kilograms. Detected by Supervision and Inspection Center of Agricultural Products of the Ministry of Agriculture, the grease content achieves 52.9%, higher than that of the non-mutated peanut by about 5 percentage points. It is proved from an experiment that the implanting survival rate is above 90%, the mutation rate is above 30%, the ideal variation rate is above 10% and the success rate of breeding is above 90%.

### Embodiment 7

In the embodiment, the wheat is used as a breeding target plant and the potato is used as an induced plant for implementing the implanting of a fission for performing the mutation breeding of the wheat, comprising the following steps:

1) planting a potato seedling until the seedling grows to 5 to 10 cm, and using a bud point of the potato seedling in a position 3 to 5 cm away from the upper part of the root as an implanting notch point;

2) selecting a wheat seeding having a seedling height of 3 to 5 cm for standby;

3) notching on the implanting notch point along the growth direction of the potato stem, wherein the size and the depth of the notch are determined according to the thickness of the wheat seedling;

4) cutting the lower part of the wheat seedling into a shape consistent with a notch and then, implanting the wheat seedling into the notch under the conditions of 11 a.m., a temperature of 30° C. and a relative humidity of 75%;

5) fixing the wheat seedling and the potato seedling with a film, covering the same with wet soil having the water content of 85%, and compacting;

6) shading and curing, and sprinkling water in a mist shape with an electric atomizer for 12 times per day;

7) after 10 to 15 days, growing 1 to 2 autogenetic roots by the wheat seedling from the upper part of the notch; up to 45 to 60 days, growing 3 to 5 autogenetic roots by the wheat seedling; and independently growing; and

8) cutting off both sides of the potato seedling notch by 5 cm; digging out a section of cut potato stem together with soil around the wheat seedling and the notch and transplanting to a prepared breeding field; and proceeding with curing growth.

12 seeds are born by the wheat in the same year. In the second year, 5 seeds are sown and survived; F1 generation is harvest; and an experimental field is sown. Compared with the non-mutated wheat, the seedling is obviously varied; the mutated wheat has a plant height of 70 to 75 cm, and the non-mutated wheat has a plant height of 80 to 85 cm; after the wheat is harvested, obvious fertile features are exhibited: large panicle type, large number of seeds, grain weight of 50 to 55 g, and 40 to 42 g for the non-mutated wheat. After planting in a peasant field, the yield per mu achieves more than 700 kilograms. Detected by Supervision and Inspection Center of Agricultural Products of the Ministry of Agriculture, the protein content achieves 16.56% (14% for the non-mutated wheat) and the vitamin content is higher than that of the non-mutated wheat. It is proved from an experiment that the implanting survival rate is above 90%, the mutation rate is above 30%, the ideal variation rate is above 10% and the success rate of breeding is above 90%.

### Embodiment 8

In the embodiment, the chili is used as a breeding target plant and the eggplant is used as an induced plant for implementing the implanting of a fission for performing the mutation breeding of the chili, comprising the following steps:

1) planting an eggplant seedling until the seedling grows to 5 to 10 cm, and using a bud point of the eggplant seedling in a position 3 to 5 cm away from the upper part of the root as an implanting notch point;

2) selecting a chili seedling having a seedling height of 3 to 5 cm for standby;

3) notching on the implanting notch point along the growth direction of the eggplant stem, wherein the size and the depth of the notch are determined according to the thickness of the chili seedling;

4) cutting the lower part of the chili seedling into a shape consistent with a notch and then, implanting the chili seedling into the notch under the conditions of 10 a.m., a temperature of 28° C. and a relative humidity of 77%;

5) fixing the chili seedling and the eggplant seedling with a film, covering the same with wet soil having the water content of 85%, and compacting;

6) shading and curing, and sprinkling water in a mist shape with an electric atomizer for about 10 times per day;

7) after 10 to 15 days, growing 1 to 2 autogenetic roots by the chili seedling from the upper part of the notch; up to about 50 days, growing 3 to 5 autogenetic roots by the chili seedling; and independently growing; and

8) cutting off both sides of the eggplant seedling notch by 5 cm; digging out a section of cut eggplant stem together with soil around the chili seedling and the notch and transplanting to a prepared breeding field; and proceeding with curing growth.

The chili blossoms and seeds in the same year as a seed. In the second year, breeding and planting are performed; compared with the non-mutated chili, the seedling is obviously varied; the mutated chili has a plant height of 75 to 80 cm, and the non-mutated chili has a plant height of 65 to 70 cm; The leaf is largened; the growing trend is vigorous; after the chili is born, the fruit shape is thickened and shortened, and the skin and flesh are thick. It is proved from an experiment that the implanting survival rate is above 90%, the mutation rate is above 30%, the ideal variation rate is above 10% and the success rate of breeding is above 90%.

### Embodiment 9

In the embodiment, the melon is used as a breeding target plant and the watermelon is used as an induced plant for implementing the implanting of a fission for performing the mutation breeding of the melon, comprising the following steps:

1) planting a watermelon seedling until the seedling grows to 10 to 15 cm, and using a bud point of the watermelon seedling in a position 3 to 5 cm away from the upper part of the root as an implanting notch point;

2) selecting a melon seedling having a seedling height of 5 to 8 cm for standby;

3) notching on the implanting notch point along the growth direction of the watermelon stem, wherein the size and the depth of the notch are determined according to the thickness of the melon seedling;

4) cutting the lower part of the melon seedling into a shape consistent with a notch and then, implanting the melon seedling into the notch under the conditions of 3 a.m., a temperature of 25° C. and a relative humidity of 81%;

5) fixing the melon seedling and the watermelon seedling with a film, covering the same with wet soil having the water content of 85%, and compacting;

6) shading and curing, and sprinkling water in a mist shape with an electric atomizer for about 10 times per day;

7) after 10 to 15 days, growing 2 to 3 autogenetic roots by the melon seedling from the upper part of the notch; up to about 50 days, growing 3 to 5 autogenetic roots by the melon seedling; and independently growing; and

8) cutting off both sides of the watermelon seedling notch by 10 cm; digging out a section of cut watermelon stem together with soil around the melon seedling and the notch and transplanting to a prepared breeding field; and proceeding with curing growth.

The melon blossoms and seeds in the same year as a seed. In the second year, breeding and planting are performed; and compared with the non-mutated melon seedling, the seedling is obviously varied; the leaf is largened; the color is dark green; the vine is long; the branches are much more; many fruit can be born; the fruit shape is round and large; the flesh is thick; and the sweet degree is increased. It is proved from an experiment that the implanting survival rate is above 90%, the mutation rate is above 30%, the ideal variation rate is above 10% and the success rate of breeding is above 90%.

### Embodiment 10

In the embodiment, the Chinese rose is used as a breeding target plant and the paeonia lactiflora pall is used as an induced plant for implementing the implanting of a fission for performing the mutation breeding of the Chinese rose, comprising the following steps:

1) planting a paeonia lactiflora pall seedling until the seedling grows to 5 to 10 cm, and using a bud point of the paeonia lactiflora pall seedling in a position 3 to 5 cm away from the upper part of the root as an implanting notch point;

2) selecting a Chinese rose seedling having a seedling height of 3 to 5 cm for standby;

3) notching on the implanting notch point along the growth direction of the paeonia lactiflora pall stem, wherein the size and the depth of the notch are determined according to the thickness of the Chinese rose seedling;

4) cutting the lower part of the Chinese rose seedling into a shape consistent with a notch and then, implanting the Chinese rose seedling into the notch under the conditions of 4 p.m., a temperature of 28° C. and a relative humidity of 77%;

5) fixing the Chinese rose seedling and the paeonia lactiflora pall seedling with a film, covering the same with wet soil having the water content of 85%, and compacting;

6) shading and curing, and sprinkling water in a mist shape with an electric atomizer for about 10 times per day;

7) after 10 to 15 days, growing 3 to 5 autogenetic roots by the Chinese rose seedling from the upper part of the notch; up to about 50 days, growing 5 to 8 autogenetic roots by the Chinese rose seedling; and independently growing; and

8) cutting off both sides of the paeonia lactiflora pall seedling notch by 5 cm; digging out a section of cut paeonia lactiflora pall stem together with soil around the Chinese rose seedling and the notch and transplanting to a prepared breeding field; and proceeding with curing growth.

The Chinese rose seedling grows and blossoms in the same year. Compared with the non-mutated Chinese rose, the petals are increased and the color is brighter; as a seed, the cut seedling blossoms in the second year; compared with the non-mutated Chinese rose, the seedling is obviously varied; the flower type is increased and the petals are denser; the color presents rainbow; and the growing trend is vigorous. It is proved from an experiment that the implanting survival rate is above 90%, the mutation rate is above 30%, the ideal variation rate is above 10% and the success rate of breeding is above 90%.

I claim:

1. A breeding method for variation of target plant for plant induced breeding, characterized by comprising the following steps:
1) breeding an induced plant seedling, and using a bud point or a node on the induced plant seedling as an implanting notch point;
2) selecting a breeding target plant seedling for standby;
3) notching on the implanting notch point;
4) cutting the lower part of the breeding target plant seedling into a shape consistent with a notch and then, implanting the breeding target plant seedling into the notch;
5) fixing the breeding target plant and an induced plant, covering the same with wet soil, and compacting;
6) shading and curing, and sprinkling water per day;
and 7) after 45 to 60 days, cutting off both sides of the induced plant notch by 5 to 10 cm; and digging out and transplanting a section of cut induced plant stem together with soil around the breeding target plant and the notch to obtain.

2. The breeding method for variation of target plant for plant induced breeding according to claim 1, characterized in that the implanting notch point in step 1) is a bud point or a node in a position 3 to 10 cm away from the root of the induced plant seedling.

3. The breeding method for variation of target plant for plant induced breeding according to claim 1, characterized in that the breeding target plant seedling in step 2) has a height of 3 to 10 cm.

4. The breeding method for variation of target plant for plant induced breeding according to claim 1, characterized in that in step 3), on the implanting notch point, notching is performed along the growth direction of the induced plant stem.

5. The breeding method for variation of target plant for plant induced breeding according to claim 1, characterized in that an implanting environment in step 4) has a temperature of 25 to 30° C. and a relative humidity of 75% to 85%.

6. The breeding method for variation of target plant for plant induced breeding according to claim 1, characterized in that the implanting time in step 4) is at 1 to 3 a.m., 8 to 11 a.m. or 3 to 6 p.m.

7. The breeding method for variation of target plant for plant induced breeding according to claim 1, characterized in that the water content of the wet soil in step 5) is 75% to 85%.

8. The breeding method for variation of target plant for plant induced breeding according to claim 1, characterized in that the sprinkling of water in step 6) is 8 to 15 times per day.
